(19) United States
(12) Patent Application Publication
(10) Pub. No.: US 2005/0255530 A1
(43) Pub. Date: Nov. 17, 2005

(54) Identification and isolation of pluripotency determining factors

(76) Inventors: Carol L. Keefer, College Park, MD (US); Steven R. Bischoff, Stow, OH (US)

Correspondence Address: ARENT FOX PLLC, 1050 CONNECTICUT AVENUE, N.W., SUITE 400, WASHINGTON, DC, 20036, US

(21) Appl. No.: 11/050,440

(22) Filed: Feb. 4, 2005

Related U.S. Application Data
(60) Provisional application No. 60/542,498, filed on Feb. 6, 2004.

(51) Int. Cl. G01N 33/53; G01N 33/567; C07H 21/04; C12N 5/06; C07K 14/475

(52) U.S. Cl. 435/7.2; 536/23.5; 435/325; 530/399

(57) ABSTRACT

Transcription factors associated with maintenance of pluripotency, Oct-4 and Nanog, are expressed in bovine and/or caprine pre-implantation embryos and isolated. Oct-4 protein and mRNA are expressed in both the inner cell mass and trophectoderm of expanded goat blastocysts. Oct-4 may play a role in trophectoderm proliferation and prevention of premature differentiation in elongating blastocysts. Nanog mRNA is expressed in the inner cell mass.

### CROSS-REFERENCE TO RELATED APPLICATIONS

This claims priority to U.S. Provisional Patent Application Ser. No. 60/542,498, filed Feb. 6, 2004, the contents of which are incorporated in their entirety herein by reference.

### BACKGROUND OF THE INVENTION

1. Field of the Invention

The present invention relates to the isolation of pluripotency transcription factors in ruminants.

2. Background of the Invention

During mammalian embryo development, initial cellular differentiation becomes readily observable during compaction and blastocyst formation. At this time, the embryonic cells become committed to two distinct developmental pathways, the trophectoderm (TE), giving rise to extraembryonic tissues, and the inner cell mass (ICM), giving rise to the definitive germ layers of the embryo. This process of cellular differentiation is characterized by distinct alterations in gene and protein expression, including transcription factors involved in determination of cell fate, cytokines involved in autocrine and paracrine signaling, and other structural and functional proteins involved in cell morphology and physiology.

An increasing number of transcription factors that are involved in the determination of cell fate at this key point in early embryonic development have been identified. Two of these transcription factors, Oct-4 and Nanog, are thought to work in concert to maintain pluripotency and self-renewal in ICM and embryonic stem (ES) cells. Oct-4, a POU octamer-binding domain transcription factor, is known to be critical in mammalian embryonic development. In the mouse, embryonic expression of Oct-4 begins at the 4- to 8-cell stage, and becomes more intensive at the morula stage embryos. Oct-4 protein is expressed at the early blastocyst stage in both ICM and TE. However, expression is rapidly down-regulated in the TE and is generally limited to the ICM cells by the expanded blastocyst stage.

It has been demonstrated that Oct-4 plays a pivotal role in establishing and maintaining cell lineage pluripotency both in vivo and in vitro. The deletion of Oct-4 causes an early lethality in the mouse at 3.5 days of gestation. A pluripotent ICM is not formed and the cells differentiate into a TE lineage. Conditional repression of Oct-4 in mouse ES cells also resulted in differentiation into trophoblast lineage, while over-expression resulted in differentiation into primitive endoderm. These studies suggested that the level of Oct-4 expression was a critical factor in the determination of cell lineage. It has been proposed that Oct-4 is necessary for maintenance of ICM pluripotency and acts, in part, by repressing trophoblast lineages in the mouse. However, Oct-4 may also be critical to trophectoderm proliferation in that Oct-4, together with the transcription factor Sox2 (50 SRY-related HMG box 2), upregulates fibroblast growth factor 4 (FGF4) expression in the ICM. FGF4 stimulates TE proliferation by a paracrine action in mouse embryos and is also necessary for maintenance of TE stem cells in vitro.

In contrast to the mouse and human, Oct-4 protein is normally expressed in the TE of fully expanded caprine, bovine, and porcine blastocysts. As caprine, bovine, and porcine embryos all show extensive proliferation and elongation of the trophectoderm and a prolonged preimplantation stage in comparison to mice and humans, continued expression of Oct-4 protein in the trophectoderm may be necessary to prevent premature differentiation.

The expression of a novel homeobox gene, Nanog, during early embryogenesis in the mouse has been reported. Nanog also plays a key role in self-renewal and maintenance of pluripotency in mouse ICM and ES cells. Deletion of the gene for Nanog is an embryonic lethal and results in the loss of pluripotency in both ICM and ES cells. Nanog deficient ICM (Nanog −/−) and ES cells differentiate into extraembryonic endoderm. Nanog protein was detected as early as the morula stage. Strikingly, Nanog was strongly expressed in the inner apolar cells, but weakly or not expressed in the outer polar cells of the late morula. At the blastocyst stage, Nanog was only expressed in the ICM and was not expressed in the TE.

Currently, Oct-4 orthologs have been identified in mouse (Genbank Accession No. MGI:101893), human (Genbank Accession No. HGNC:9221), rat, bovine (Genbank Accession No. AY490804), and pig, while Nanog orthologs have been identified in mouse (Genbank Accession No. AY278951), human (Genbank Accession No. NM_{—}024865), and rat.

In addition to the pluripotency-determining factor Oct-4, stage-specific embryonic antigens (SSEA1, SSEA3, and SSEA4), and tumor rejection antigens (TRA-1-60 and TRA-1-81) have been widely used as markers to identify ES cell and monitor differentiation in the mouse and human. The expression of these various surface antigens on mouse and human ES cells correspond to those observed on the ICM. In domestic animals, SSEA1 is expressed in porcine ES-like cells, and bovine ES-like cells express SSEA1, SSEA3 and SSEA4. However, little information is available on the expression patterns of these ES cell surface markers in preimplantation embryos of domestic animals. Systematical investigations on the expression patterns of the key pluripotency transcription factors and ES cell surface antigens in preimplantation embryos from domestic animal could provide vital information on early embryo development and aid in the derivation of ES cells in these species.

In the present invention, expression patterns of Oct-4, Nanog, SSEA1, SSEA4, TRA-1-60 and TRA-1-81 were analyzed in parthenogenetic and in vivo derived goat embryos at the 8-cell, morula and blastocyst stages. Sox2, FGF4, and fibroblast growth factor receptor 2 (FGFR2) were analyzed in ICM and TE of parthenogenetic and in vivo derived blastocysts.

There has been no evidence that Oct-4 and Nanog are expressed during early embryogenesis in the goat. Moreover, no prior art has identified Nanog expression in domestic animals. Accordingly, there is an unmet need in the art to determine whether Oct-4 and Nanog are expressed in various mammalian embryos. There are also unmet needs to identify and isolate the expression of Oct-4 and Nanog in ruminants.

### SUMMARY OF THE INVENTION

One aspect of the present invention is directed to identifying and isolating pluripotency factors in mammals. Mammals of particular interest include ruminants, and more particularly goat.

Another aspect of the present invention is directed to identifying and isolating genes encoding pluripotency factors in mammals, more particularly ruminants.

Yet another aspect of the present invention is directed to pluripotency factors in mammals.

One other aspect of the present invention is directed to identifying and isolating polynucleotides comprising the nucleotide sequence of Oct-4 and Nanog.

A further aspect of the present invention is directed to identifying and isolating a polynucleotide encoding polypeptides comprising the amino acid sequences of Oct-4 and Nanog.

One more aspect of the present invention is directed to identifying and isolating a polynucleotides complementary to Oct-4 and Nanog under certain stringency conditions of from 1×SSC to 10×SSC.

Yet one other aspect of the present invention is directed to a method of identifying at least one ruminant pluripotency factor comprising: developing a ruminant embryo; incubating the ruminant embryo with a monoclonal antibody, wherein the monoclonal antibody is specific to a non-ruminant pluripotency factor and is capable of forming an antibody complex with a corresponding ruminant pluripotency factor; and detecting the antibody complex to identify the ruminant pluripotency factor.

Yet a further aspect of the present invention is directed to identifying and isolating sense and antisense primer sequences of Nanog and Oct-4.

Additional advantages and novel features of the invention will also become more apparent to those skilled in the art upon examination of the following or upon learning by practice of the invention.

### BRIEF DESCRIPTION OF THE DRAWINGS

FIGS. 1A-1I illustrate the expression of Oct-4 versus a negative and background control;

FIGS. 2A and 2B present the expression of Oct-4 and Nanog with respect to stages of embryonic development;

FIG. 3 presents a portion of goat Nanog nucleotide sequence as compared to human Nanog;

FIG. 4 presents the expression of certain cell markers in in vivo and parthenogenetic embryos in various development stages;

FIG. 5 presents goat OCT-4 DNA sequence;

FIG. 6 presents the predicted goat OCT-4 amino acid sequence;

FIG. 7 presents the goat Nanog DNA sequence; and

FIG. 8 presents the predicted goat Nanog amino-acid sequence.

Other features of the present invention will become apparent from the following detailed description considered in connection with the accompanying drawings. It should be understood, however, that the figures are designed for the purpose of illustration only and not as a definition of the limits of the invention. Additional advantages and novel features of the invention will also become apparent to those skilled in the art upon examination of the following or upon learning by practice of the invention.

### DETAILED DESCRIPTION

The present invention describes the discovery and isolation of pluripotency factors from domestic animal blastocysts. Embodiments include blastocysts of bovine and caprine animals. Specifically, the present invention is directed to discovery and isolation of pluripotency transcription factors Oct-4 and Nanog. The present invention meets the needs in the art by analyzing the expression patterns of Oct-4 and Nanog in parthenogenetic and in vivo derived caprine and/or bovine embryos at the 8-cell, morula and blastocyst stages. In a preferred embodiment, the transcription factors Oct-4 and Nanog have homologies to their respective human sequences.

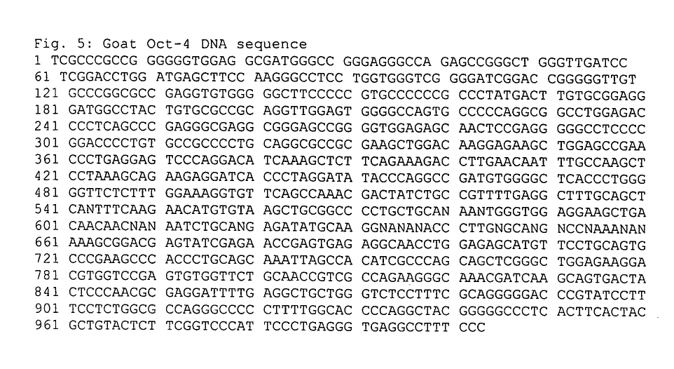

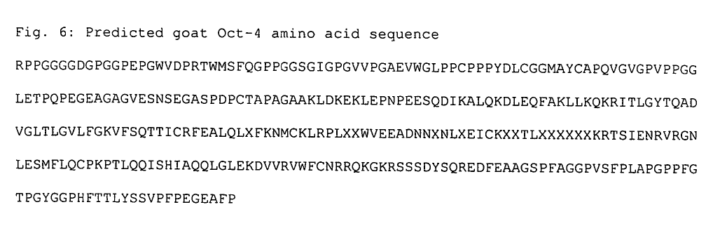

As shown in FIG. 5, a 1003 base pair product (SEQ ID NO:1) is created by using RT-PCR Oct-4 primers. Oct-4 primers are designed based on the bovine open reading frame (Genbank Accession No. NM_{—}174580). The sense primer used is 5′-ATGGCGGGACACCTCGCTTCTCCCCAAAGC-3′ (SEQ ID NO:5). The antisense primer used is 5′-TCAGTTTGCATGCATAGGGGAGCCCAGAG C-3′ (SEQ ID NO:6). SEQ ID NO:1 encodes an amino acid sequence, SEQ ID NO:2, which consists of 333 amino acids, as shown in FIG. 6.

One aspect of the present invention comprises a nucleotide sequence that has a homology selected from 100%, 99%, 98%, or 97% to SEQ ID NO:1. In one embodiment, this percentage homology is with respect to human Oct-4 nucleotide sequences, DNA and/or RNA. In another embodiment, this percentage homology is with respect to mouse Oct-4. In yet another embodiment, this percentage homology is with respect to monkey Oct-4. In one more embodiment, this percentage homology is with respect to bovine Oct-4.

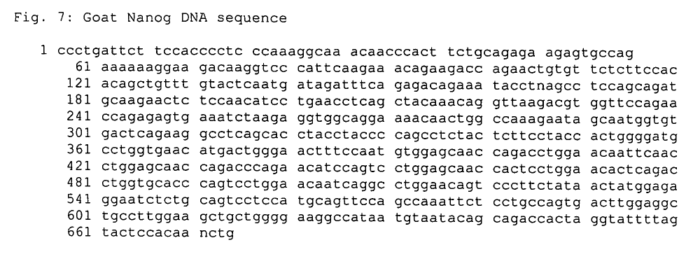

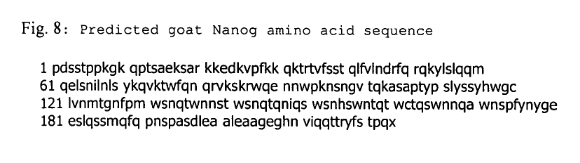

As shown in FIG. 7, a 674 base pair product (Genbank Accession No. AY786437) (SEQ ID NO:3) in goat (Capra hircus) is created by RT-PCR using Nanog primers. Nanog primers were designed based on human Nanog sequence (Genbank Accession No. 024865). The sense primer used is 5′-CAGCCCTGATTCTTCCACCAGTCCCAAAGGCM-3′ (SEQ ID NO:7). The antisense primer used is 5′-GAGTAGTTTAGGAATAAATCCA-3′ (SEQ ID NO:8). SEQ ID NO:3 encodes an amino acid sequence (Genbank Accession No. AAW50709), SEQ ID NO:4, which consists of 224 amino acids, as shown in FIG. 8.

It is one aspect of the present invention to provide a nucleotide sequence that has a homology selected from 100%, 99%, 98%, 97%, 96%, 95%, or 94% to SEQ ID NO:3. In one embodiment, this percentage homology is with respect to human Nanog nucleotide sequences, DNA and/or RNA. In another embodiment, this percentage homology is with respect to mouse Nanog. In yet another embodiment, this percentage homology is with respect to rat Nanog. In one more embodiment, this percentage homology is with respect to macaque Nanog.

The present invention also covers replacement of between 1 and 20 nucleotides of either SEQ ID NO:1 or SEQ ID NO:3, with non-natural or non-standard nucleotides for example phosphorothioate, deoxyinosine, deoxyuridine, isocytosine, isoguanosine, ribonucleic acids including 2-O-methyl, and replacement of the phosphodiester backbone with, for example, alkyl chains, aryl groups, and protein nucleic acid (PNA).

It is another aspect of the present invention to provide a nucleotide sequence that hybridizes to either SEQ ID NO:1 or SEQ ID NO:3 under a stringency condition of 1×SSC. It is another aspect of the present invention to provide a nucleotide sequence that hybridizes to either SEQ ID NO:1 or SEQ ID NO:3 under a stringency condition of 2×SSC. It is another aspect of the present invention to provide a nucleotide sequence that hybridizes to either SEQ ID NO:1 or SEQ ID NO:3 under a stringency condition of any one of 3×SSC. It is another aspect of the present invention to provide a nucleotide sequence that hybridizes to either SEQ ID NO:1 or SEQ ID NO:3 under a stringency condition of 4×SSC. It is another aspect of the present invention to provide a nucleotide sequence that hybridizes to either SEQ ID NO:1 or SEQ ID NO:3 under a stringency condition of 5×SSC. It is another aspect of the present invention to provide a nucleotide sequence that hybridizes to any one of either SEQ ID NO:1 or SEQ ID NO:3 under a stringency condition of 6×SSC. It is another aspect of the present invention to provide a nucleotide sequence that hybridizes to either SEQ ID NO:1 or SEQ ID NO:3 under a stringency condition of 7×SSC. It is another aspect of the present invention to provide a nucleotide sequence that hybridizes to either SEQ ID NO:1 or SEQ ID NO:3 under a stringency condition of 8×SSC. It is another aspect of the present invention to provide a nucleotide sequence that hybridizes to either SEQ ID NO:1 or SEQ ID NO:3 under a stringency condition of 9×SSC. It is another aspect of the present invention to provide a nucleotide sequence that hybridizes to either SEQ ID NO:1 or SEQ ID NO:3 under a stringency condition of 10×SSC.

It is another aspect of the present invention to provide a nucleotide sequence that encodes a polypeptide. It is well understood that due to the degeneracy of the genetic code, an amino acid can be coded for by more than one codon. Therefore, the present invention encompasses all polynucleotides that code for either SEQ ID NO:2 or SEQ ID NO:4.

The scope of this invention covers natural and non-natural alleles of either SEQ ID NO:2 or SEQ ID NO:4. In a preferred embodiment of the present invention, alleles of either SEQ ID NO:2 or SEQ ID NO:4 comprise replacement of one, two, three, four, or five naturally occurring amino acids with similarly charged, shaped, sized, or situated amino acids (conservative substitutions). The present invention also covers non-natural or non-standard amino acids for example selenocysteine, pyrrolysine, 4-hydroxyproline, 5-hydroxylysine, phosphoserine, phosphotyrosine, and the D-isomers of the 20 standard amino acids.

One embodiment of the present invention comprises identification and isolation of pluripotency factors in ruminants. In general, discovery and isolation of such pluripotency transcription factors involves selecting an embryo of a desired animal, culturing it and developing it to a particular stage of development. In an embodiment of the present invention, embryos of domestic animals, such including caprine, porcine, and bovine, are selected, cultured, and developed.

Isolation and identification are generally conducted in the following manner. Embryos are fixed at certain stages of embryonic development, such as 8-cell, morula and blastocysts, using methods known or understood to one skilled in the art.

One such method includes immunohistochemical analysis, which involves exposure of the ruminant embryos to relevant antibodies. Relevant antibodies include antibodies specific to a pluripotency factor in non-ruminant animals that are capable of forming an antibody complex with a corresponding ruminant pluripotency factor.

For example, ruminant embryos, which can contain ruminant pluripotency factors and their corresponding proteins, are exposed to antibodies specific to a corresponding pluripotency factor in a non-ruminant, which form an antibody complex if the target pluripotency factor is found in the embryo. Non-ruminant antibodies are generally obtained from animals in which the pluripotency factor to be examined (e.g., Oct-4, Nanog, etc.) has been isolated and an antibody complex is known to form when the antibody is exposed to the pluripotency factor. In the case of Oct-4, for example, mouse antibodies, which are known to be reactive to human Oct-4, are exposed to ruminant embryos in the expectation that the mouse antibodies are cross-reactive to ruminant Oct-4. Accordingly, antibody complexes form when the pluripotency factor is present in the embryo. In a preferred embodiment of the present invention, human antibodies, which are known to be reactive to human pluripotency factors are used in the same way as the mouse antibodies described above.

The antibody complex is then detected. For example, the antibody complex may be detected by washing, staining and mounting the embryos on slides.

Replication and amplification of the relevant nucleotides is conducted as understood in the art. With respect to RT-PCR and sequencing analysis, RNA is extracted from embryos at various stages. In one embodiment, specific primers for Oct-4, Nanog, Sox2, FGF4, and FGFR2 are used.

Sox2 primers were designed based on homology between human and mouse Sox2 open reading frame. The sense primer used is 5′-AAGATGCACMCTCGGAGATCA-3′ (SEQ ID NO:9). The antisense primer used is 5′-AGCCGTTCATGTAGGTCTGCG A-3′ (SEQ ID NO:10).

The invention covers FGF4 primers, for example, the sense primer: 5′-TTCTTCGTGGCCATGAGCAG-3′ (SEQ ID NO:11), and the antisense primer: 5′-AGGMGTGGGTGACCTTCAT-3′ (SEQ ID NO:12).

The invention also covers FGFR2 primers such as the sense primer: 5′-GTCATCGTTGAATACGCCTC-3′ (SEQ ID NO:13), and the antisense primer: 5′-TCTGATGGGTGTACACTCTG-3′ (SEQ ID NO:14).

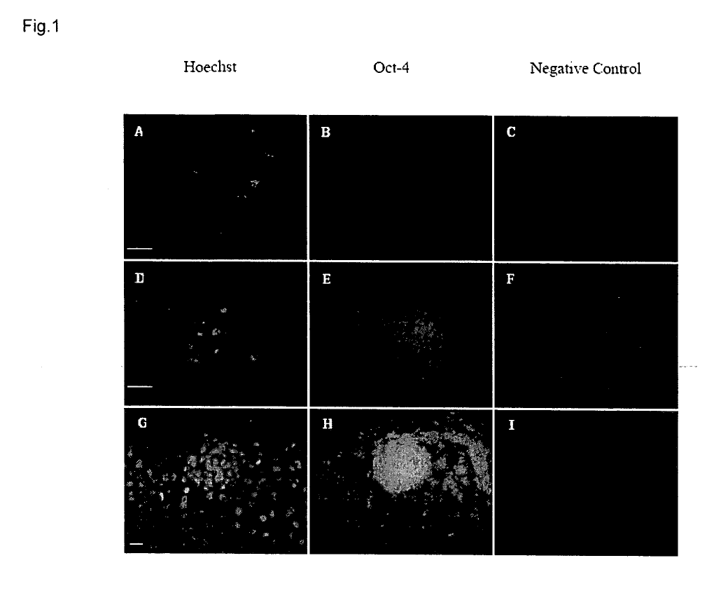

Expression of Oct-4 mRNA is detected at three embryo stages: 8-cell, morula, and blastocyst. Oct-4 protein expression is first detected at the morula stage, as shown in FIGS. 1D-1F. The Oct-4 signal is distributed in both cytoplasm and nuclei at the morula stages. A stronger signal is observed at the blastocyst stage, as shown in FIGS. 1G-1I. The Oct-4 signal moved from a cytoplasmic location at the morula stage to a nuclear location at the blastocyst stage. Both the ICM and TE are positive for Oct-4 staining in day 7 and day 10 blastocysts.

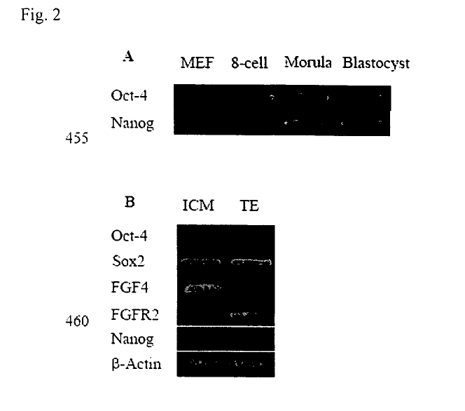

As shown in FIG. 2A, Oct-4 mRNA was detected at 8-cell, morula and blastocyst stages. Both the ICM and TE of goat blastocysts expressed Oct-4 mRNA, as shown in FIG. 2B. No amplification products were obtained from the negative control, mouse embryonic fibroblasts. DNA sequencing confirmed that the PCR products produced with Oct-4 primers had 96% homology to the bovine sequence.

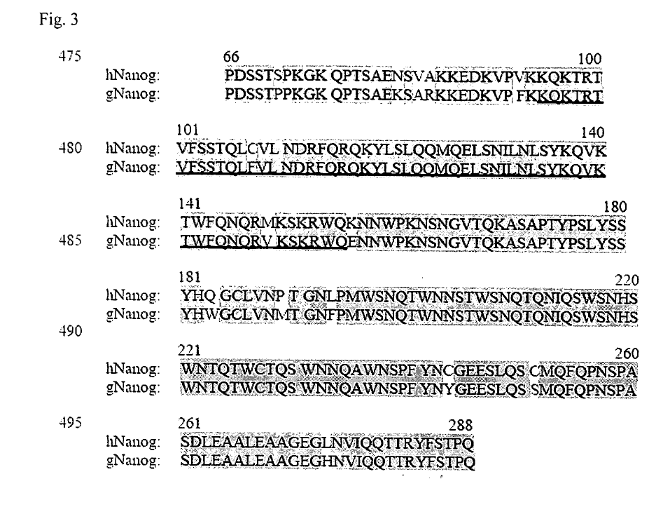

Nanog mRNA was found in morulae and blastocysts, but not in 8-cell embryos, as shown in FIG. 2A. In contrast to Oct-4 expression pattern, Nanog mRNA was only detected in the ICM and not in the TE. DNA sequencing revealed a 93% homology to human sequence, as shown in FIG. 3.

Sox2 and FGF4 mRNA were detected in the ICM and TE, and FGFR2 mRNA was expressed in the TE, as shown in FIG. 2B.

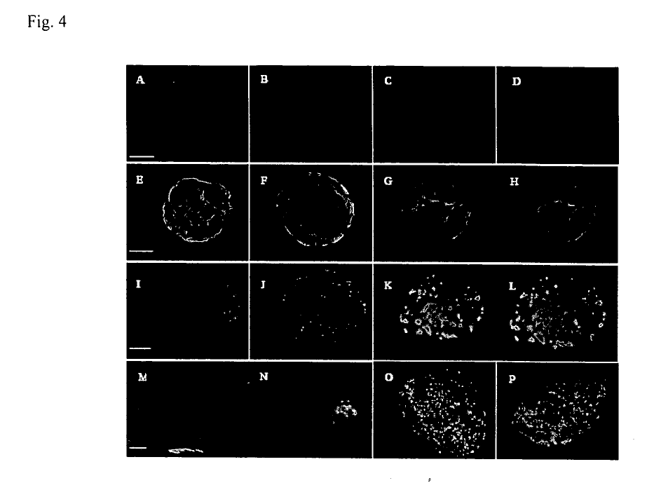

There is no difference in expression of any of four cell surface markers was detected between in vivo- and parthenogenetic embryos, although in vivo embryos generally were of better quality (morphology, cell number) and had a more consistent, even staining pattern than parthenogenetic embryos. No difference in expression of any of the four cell surface makers was detected between in vivo (FIGS. 4A-4L) and parthenogenetic embryos (FIGS. 4M-4P), although in vivo embryos generally were of better quality (morphology, cell number) and had a more consistent, even staining pattern than parthenogenetic embryos. Strong expression of SSEA4 was shown at 8-cell (FIG. 4E), morula (FIG. 4F), and blastocyst stages (FIGS. 4 G-4H). In contrast, SSEA1 signal was weak at the 8-cell stage (not shown), but became stronger in blastocysts (FIG. 4N).

At the blastocyst stage, SSEA1 was detected in both the ICM and the TE of in vivo derived blastocysts. Parthenogenetic embryos strongly expressed SSEA1 in the ICM and on dividing or recently divided cells of the TE. Both TRA-1-60 and TRA-1-81 were detected on the inner surface of the zona pellucida and within the perivitelline space at all three embryonic stages examined (FIGS. 4I-4L). Day 7 and 10 hatched, expanded blastocysts displayed a similar, punctate staining that appeared to be on, or external to, the cell surface (FIGS. 4O-4P).

The present invention shows that Oct-4 expression pattern in preimplantation embryos, including onset and distribution, varies among different species. While the onset of Oct-4 protein expression has been reported at the 4- to 8-cell stage in the mouse and at the 16-cell stage in the monkey, it has not been detectable until the late morula stage (>32 cells) in ruminants. This late detection of Oct-4 protein in goat embryos is unlikely due to a low affinity between goat Oct-4 and the primary antibody since strong signals were detected at the blastocyst stage. In the present invention, Oct-4 mRNA is detectable at all stages from GV oocytes through to blastocysts in the mouse and bovine.

Oct-4 protein was not expressed until after the maternal-zygotic transition (MZT). Data showing onset of Oct-4 protein expression at the morula stage in the goat are consistent with the MZT occurring at the 8- to 16-cell stage in ruminants. The distribution pattern of Oct-4 protein and mRNA in goat pre-implantation embryos is consistent with that observed in bovine and porcine, in which Oct-4 protein has been shown to be present in both cytoplasm and nuclei at the morula and blastocyst stages and was expressed in both the ICM and the TE. Oct-4 protein and mRNA expression in the TE of goat blastocysts at day 10 were maintained at levels comparable to the ICM. This finding is consistent with the report that Oct-4 protein was present in the TE cells of hatched bovine blastocysts at Day 10.

In contrast, in the mouse and monkey Oct-4 protein expression in the TE was down regulated rapidly after formation of the blastocyst and was restricted to the ICM in the expanded blastocysts. In the human blastocysts, Oct-4 mRNA expression level in the ICM has been shown to be 31-fold higher than that in the TE. In mice and humans, Oct-4 is considered a major repressor of the TE and the trophoblast lineage. In Oct-4 null mutant mice, the ICM differentiated into trophoblast lineages and conditionally repressed Oct-4 expression triggered mouse ESC differentiation into trophoblast giant cells. Furthermore, the expression of several trophoblast-specific genes was directly repressed by Oct-4.

The present invention also includes the continued expression of Oct-4 in TE of goat, bovine and porcine blastocysts. Oct-4 expression in the TE may be related to the longer period of TE proliferation before implantation in those domestic animals.

The role of Oct-4 in TE proliferation is related to whether Sox2 and the growth factor FGF4, a target gene upregulated by Oct-4 and Sox2. In the mouse, FGF4 expression is restricted to the ICM, however, it is required for TE development through paracrine actions in vivo, and is a key factor in maintaining trophoblast stem cells in vitro. The presence of Sox2 and FGF4 in both goat TE and ICM supports the hypothesis that Oct-4 expression in the TE is required to stimulate FGF4, and thus, to help maintain TE cell proliferation and prevent premature differentiation. Goat TE, but not ICM, also expresses FGFR2, which can act as the receptor for FGF4. In contrast to mice, ruminant and porcine embryos elongate dramatically before implantation. The size of the early embryonic vesicle (for example, ˜25 cm in length at day 17 for bovine blastocysts) dictates that most TE are far away from the ICM. Thus, FGF4 secreted by the ICM may not be enough to maintain TE proliferation through paracrine action as it does in the mouse.

Expression patterns of Oct-4 described herein are useful to demonstrate that a transcription factor may have different expression patterns in ruminants than expression patterns already known in the mouse. Oct-4 expression in both the ICM and the TE points to the need to identify a transcription factor with a pattern of expression that is restricted to the ICM, i.e., Nanog.

Non-limiting examples of experimental methods used in the present invention are described.

### EXAMPLE 1

### In Vivo Embryo Collection

The herds of pure- and mixed-breed, scrapie-free, Alpine, Saanen, and Toggenburg dairy goats used for this study were maintained under Good Agricultural Practice (GAP) guidelines at the GTC Biotherapeutics farm in Massachusetts. Embryo donor does, synchronized and superovulated as previously described, were naturally breed with intact bucks, and/or artificially inseminated with fresh-collected semen. After collection, embryos were cultured in equilibrated M199 (GIBCO) with 10% FBS supplemented with 2 mM L-glutamine and 1% penicillin/streptomycin (10,000 IU/ml each) and shipped in a temperature controlled container (Biotherm portable incubator, Cryologics, Australia) to the University of Maryland by overnight courier.

### EXAMPLE 2

### Oocyte Maturation, Activation and Parthenogenetic Embryo Culture

Unless otherwise indicated all chemicals used in embryo culture were obtained from Sigma-Aldrich, St. Louis, Mo., USA. Oocyte maturation medium was TCM199H medium (GIBCO) supplemented with 0.02 units/ml bLH (Sioux Biochemicals, Sioux Center, Iowa), 0.002 units/ml bFSH (Sioux Biochemicals, Sioux Center, Iowa), 1 μg/ml estradiol-17β, 0.2 mM sodium pyruvate, 50 μg/ml gentamycin, 100 μM cysteamine, and 10% heat inactivated goat serum.

Ovaries were collected at slaughter and transported to the laboratory in warm (33-38° C.) PBS. Oocytes-cumulus complexes (OCC) were obtaining by slicing the ovarian surface with a razorblade. OCC with multiple cumulus layers were selected and washed three times in pre-warmed maturation medium. OCC selected were 115 120 125 130 135 incubated for 28 h at 38.5° C., in 5% CO₂. Mature metaphase II oocytes were subsequently activated by incubation with 5 μM ionomycin for 4 min, followed by incubation for 4 min in high BSA (30 mg/ml) and subsequently cultured in G1.3 medium (Vitrolife, Englewood, Colo.), supplemented with 2 mM 6-dimethylaminopurine (DMAP) at 38.5° C., in 5% CO₂ for 4 h. Oocytes were then transferred to fresh G1.3 medium, and cultured at 38.5° C. in 5% CO₂, 5% O₂, and 90% N₂. Zygotes developed into 8-cell stage after 72 h. Morulae and blastocysts were obtained by additional culture in G2.3 medium (Vitrolife, Englewood, Colo.) for 48 and 96 h, respectively.

### EXAMPLE 3

### Immunohistochemical Analysis

At least 6 in vivo- and parthenogenetic-derived goat embryos were examined at the 8-cell, morula and blastocyst (both day 7 and day 10) stages. Embryos were fixed in 4% formaldehyde for 20 min and washed three times with TBST (20 mM Tris-HCl, 0.15 M NaCl, and 0.05% Tween-20, pH 7.4, Sigma-Aldrich). After permeabilization with 0.2% Triton X-100 and 0.1% Tween-20 for 10 min, nonspecific reactions were blocked with 10% normal sheep serum (Sigma-Aldrich) for 30 min at room temperature. Embryos were then incubated overnight at 4° C. in mouse monoclonal antibodies, including Oct-4 (1:40; Santa Cruz Biotechnology, Inc., Santa Cruz, Calif.), SSEA1, SSEA4, TRA-1-60 and TRA-1-81 (1:50; ES cell characterization kit, Chemicon, Temecula, Calif.). After extensive washing with TBST buffer, embryos were exposed to sheep anti-mouse secondary antibody conjugated with FITC (1:30; Chemicon, Temecula, Calif.) for 30 min at room temperature. Either the primary antibody was omitted or normal sheep IgG conjugated with FITC (Santa Cruz Biotechnology, Inc., Santa Cruz, Calif.) was used in negative controls. Embryos 140 145 150 155 160 were then washed and stained with 1 μg/ml of Hoechst 33342 (Sigma, St. Louis, Mo.) for 10 min, and whole-mounted onto slides. Samples were examined with a Leica DM IRE2 inverted microscope (Vashaw Scientific, Inc., Frederick, Md.).

### EXAMPLE 4

### RT-PCR and Sequencing Analysis

Total RNA was extracted from 8-cell, morulae, whole blastocysts, ICM and TE using an Absolutely RNA Nanoprep Kit (Stratagene, La Jolla, Calif.) according to the manufacturer's instructions. ICM and TE were visualized by microscopy and manually dissected. Non-reverse-transcribed RNA was included as a control for genomic DNA contamination for each RNA preparation. The first strand cDNAs were synthesized using superscript III (Invitrogen, Carlsbad, Calif.) and cDNAs were amplified with PfuUltra™ hotstart PCR master mix (Stratagene, La Jolla, Calif.) according to the manufacturer's instructions. Primers are designed accordingly.

The PCR thermocycling conditions were the following: an initial denaturation step at 95° C. for 3 min followed by 35 cycles of 95° C. for 30 sec, primer specific annealing temperature (65° C. for Oct-4, 50° C. for Nanog, 57° C. for FGF4, and 54° C. for FGFR2) for 30 sec, and 72° C. for 45 sec with a final extension at 72° C. for 10 min.

Positive controls of PCR reactions were carried out using primers for β-actin (sense, 5′-ATGGTGGGTATGGGTCAGM-3′ (SEQ ID NO:15); antisense, 5′-CGGAGCTCGTTGTAG AAGGT-3′ (SEQ ID NO:16)). The PCR products were separated by electrophoreses through a 2% Tris-acetate-EDTA agarose gel stained with GelStar™ (Cambrex Bio Science, Rockland, Me.) according to the manufacturer's instructions. Gels were visualized on an ultraviolet transilluminator and imaged with a digital camera (Sony, Japan). To retrieve amplified PCR products of expected size, DNA bands were excised from the gel and extracted with Qiaquick™ gel extraction kit (Qiagene, Valencia, Calif.) according to the manufacturer's instructions. The extracted DNA was sequenced by the sequencing facility at University of Maryland (College Park, Md.).

Having thus described presently preferred embodiments of the present invention, it will be appreciated that the objects of the invention have been achieved, and it will be understood by those skilled in the art that changes in construction and widely differing embodiments and applications of the invention will suggest themselves without departing from the spirit and scope of the present invention. The disclosure and description herein are intended to be illustrative and are not in any sense limiting of the invention.

1. An isolated polynucleotide comprising the nucleotide sequence of SEQ ID NO:3.

2. An isolated polynucleotide encoding a polypeptide comprising the amino acid sequence of SEQ ID NO:4.

3. An isolated polypeptide comprising the amino acid sequence of SEQ ID NO:4.

4. An isolated polynucleotide complementary to SEQ ID NO:3 under a stringency condition of from 1×SSC to 10×SSC.

5. A method of identifying at least one ruminant pluripotency factor, comprising:
developing a ruminant embryo;
incubating the ruminant embryo with a monoclonal antibody, wherein the monoclonal antibody is specific to a non-ruminant pluripotency factor and is capable of forming an antibody complex with a corresponding ruminant pluripotency factor; and
detecting the antibody complex to identify the ruminant pluripotency factor.

6. The method of claim 5, wherein the ruminant pluripotency factor is Oct-4.

7. The method of claim 5, wherein the ruminant pluripotency factor is Nanog.

8. The method of claim 5, wherein the ruminant is a caprine animal.

9. The method of claim 5, wherein the ruminant is a bovine animal.

10. The method of claim 5, wherein the ruminant embryo is developed to the 8-cell stage.

11. The method of claim 5, wherein the ruminant embryo is developed to the morula stage.

12. The method of claim 5, wherein the ruminant embryo is developed to the blastocyst stage.

13. The method of claim 5, wherein the ruminant embryo is developed to the inner cell mass stage.

14. The method of claim 5, wherein the ruminant embryo is developed to the trophectoderm stage.

15. The method of claim 5, further comprising:
isolating the ruminant pluripotency factor.

16. An isolated polynucleotide comprising the nucleotide sequence of SEQ ID NO:5.

17. An isolated polynucleotide comprising the nucleotide sequence of SEQ ID NO:6.

18. An isolated polynucleotide comprising the nucleotide sequence of SEQ ID NO:7.

19. An isolated polynucleotide comprising the nucleotide sequence of SEQ ID NO:8.

20. The method of claim 5, wherein detecting comprises staining the embryo.


SEQUENCE LISTING (18 sequences)

SEQ ID NO: 1 - DNA, 1003 bp, Capra hircus
  modified_base (543): a, c, g, or t
tcgcccgccggggggtggaggcgatgggccgggagggccagagccgggctgggttgatcctcggacctggatgagcttccaagggcctcctggtgggtcggggatcggaccgggggttgtgcccggcgccgaggtgtgggggcttcccccgtgccccccgccctatgacttgtgcggagggatggcctactgtgcgccgcaggttggagtggggccagtgcccccaggcggcctggagacccctcagcccgagggcgaggcgggagccggggtggagagcaactccgagggggcctccccggacccctgtgccgcccctgcaggcgccgcgaagctggacaaggagaagctggagccgaaccctgaggagtcccaggacatcaaagctcttcagaaagaccttgaacaatttgccaagctcctaaagcagaagaggatcaccctaggatatacccaggccgatgtggggctcaccctgggggttctctttggaaaggtgttcagccaaacgactatctgccgttttgaggctttgcagctcantttcaagaacatgtgtaagctgcggcccctgctgcanaantgggtggaggaagctgacaacaacnanaatctgcangagatatgcaaggnananacccttgngcangnccnaaananaaagcggacgagtatcgagaaccgagtgagaggcaacctggagagcatgttcctgcagtgcccgaagcccaccctgcagcaaattagccacatcgcccagcagctcgggctggagaaggacgtggtccgagtgtggttctgcaaccgtcgccagaagggcaaacgatcaagcagtgactactcccaacgcgaggattttgaggctgctgggtctcctttcgcagggggacccgtatcctttcctctggcgccagggcccccttttggcaccccaggctacgggggccctcacttcactacgctgtactcttcggtcccattccctgagggtgaggcctttccc

SEQ ID NO: 2 - protein, 333 aa, Capra hircus
  MOD_RES (181): Variable amino acid
RPPGGGGDGPGGPEPGWVDPRTWMSFQGPPGGSGIGPGVVPGAEVWGLPPCPPPYDLCGGMAYCAPQVGVGPVPPGGLETPQPEGEAGAGVESNSEGASPDPCTAPAGAAKLDKEKLEPNPEESQDIKALQKDLEQFAKLLKQKRITLGYTQADVGLTLGVLFGKVFSQTTICRFEALQLXFKNMCKLRPLXXWVEEADNNXNLXEICKXXTLXXXXXXKRTSIENRVRGNLESMFLQCPKPTLQQISHIAQQLGLEKDVVRVWFCNRRQKGKRSSSDYSQREDFEAAGSPFAGGPVSFPLAPGPPFGTPGYGGPHFTTLYSSVPFPEGEAFP

SEQ ID NO: 3 - DNA, 674 bp, Capra hircus
  modified_base (166): a, c, g, or t
ccctgattcttccacccctcccaaaggcaaacaacccacttctgcagagaagagtgccagaaaaaaggaagacaaggtcccattcaagaaacagaagaccagaactgtgttctcttccacacagctgtttgtactcaatgatagatttcagagacagaaatacctnagcctccagcagatgcaagaactctccaacatcctgaacctcagctacaaacaggttaagacgtggttccagaaccagagagtgaaatctaagaggtggcaggaaaacaactggccaaagaatagcaatggtgtgactcagaaggcctcagcacctacctaccccagcctctactcttcctaccactggggatgcctggtgaacatgactgggaactttccaatgtggagcaaccagacctggaacaattcaacctggagcaaccagacccagaacatccagtcctggagcaaccactcctggaacactcagacctggtgcacccagtcctggaacaatcaggcctggaacagtcccttctataactatggagaggaatctctgcagtcctccatgcagttccagccaaattctcctgccagtgacttggaggctgccttggaagctgctggggaaggccataatgtaatacagcagaccactaggtattttagtactccacaanctg

SEQ ID NO: 4 - protein, 224 aa, Capra hircus
  MOD_RES (224): Variable amino acid
PDSSTPPKGKQPTSAEKSARKKEDKVPFKKQKTRTVFSSTQLFVLNDRFQRQKYLSLQQMQELSNILNLSYKQVKTWFQNQRVKSKRWQENNWPKNSNGVTQKASAPTYPSLYSSYHWGCLVNMTGNFPMWSNQTWNNSTWSNQTQNIQSWSNHSWNTQTWCTQSWNNQAWNSPFYNYGEESLQSSMQFQPNSPASDLEAALEAAGEGHNVIQQTTRYFSTPQX

SEQ ID NO: 5 - DNA, 30 bp, Artificial Sequence
  Description of Artificial Sequence Synthetic primer
atggcgggacacctcgcttctccccaaagc

SEQ ID NO: 6 - DNA, 30 bp, Artificial Sequence
  Description of Artificial Sequence Synthetic primer
tcagtttgcatgcataggggagcccagagc

SEQ ID NO: 7 - DNA, 33 bp, Artificial Sequence
  Description of Artificial Sequence Synthetic primer
cagccctgattcttccaccagtcccaaaggcaa

SEQ ID NO: 8 - DNA, 22 bp, Artificial Sequence
  Description of Artificial Sequence Synthetic primer
gagtagtttaggaataaatcca

SEQ ID NO: 9 - DNA, 22 bp, Artificial Sequence
  Description of Artificial Sequence Synthetic primer
aagatgcacaactcggagatca

SEQ ID NO: 10 - DNA, 22 bp, Artificial Sequence
  Description of Artificial Sequence Synthetic primer
agccgttcatgtaggtctgcga

SEQ ID NO: 11 - DNA, 20 bp, Artificial Sequence
  Description of Artificial Sequence Synthetic primer
ttcttcgtggccatgagcag

SEQ ID NO: 12 - DNA, 20 bp, Artificial Sequence
  Description of Artificial Sequence Synthetic primer
aggaagtgggtgaccttcat

SEQ ID NO: 13 - DNA, 20 bp, Artificial Sequence
  Description of Artificial Sequence Synthetic primer
gtcatcgttgaatacgcctc

SEQ ID NO: 14 - DNA, 20 bp, Artificial Sequence
  Description of Artificial Sequence Synthetic primer
tctgatgggtgtacactctg

SEQ ID NO: 15 - DNA, 20 bp, Artificial Sequence
  Description of Artificial Sequence Synthetic primer
atggtgggtatgggtcagaa

SEQ ID NO: 16 - DNA, 20 bp, Artificial Sequence
  Description of Artificial Sequence Synthetic primer
cggagctcgttgtagaaggt

SEQ ID NO: 17 - protein, 223 aa, Homo sapiens
PDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGHNVIQQTTRYFSTPQ

SEQ ID NO: 18 - protein, 223 aa, Capra hircus
PDSSTPPKGKQPTSAEKSARKKEDKVPFKKQKTRTVFSSTQLFVLNDRFQRQKYLSLQQMQELSNILNLSYKQVKTWFQNQRVKSKRWQENNWPKNSNGVTQKASAPTYPSLYSSYHWGCLVNMTGNFPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQ